(19) United States
(12) Patent Application Publication
(10) Pub. No.: US 2012/0227735 A1
(43) Pub. Date: Sep. 13, 2012

(54) NEBULIZER FOR A AEROSOLTHERAPY

(75) Inventors: Luigi Abate, S. Martino Della Battaglia Brescia (IT); Riccardo Abate, S. Martino Della Battaglia Brescia (IT)

(73) Assignee: FLAEM NUOVA S.P.A., S. Martino Della Battaglia, Brescia (IT)

(21) Appl. No.: 13/502,007

(22) PCT Filed: Sep. 22, 2010
(86) PCT No.: PCT/IT2010/000400

§ 371 Date: May 18, 2012

(30) Foreign Application Priority Data
Oct. 15, 2009 (IT) BS2009U000030

(51) Int. Cl. A61M 11/00

(52) U.S. Cl. 128/200.14

(57) ABSTRACT

This invention concerns an ampoule nebulizer per aerosol therapy at different medicament delivery speeds. It comprises a body with a through conduit (30) for the delivery of ambient air from outside. The though conduit is associated with a selection push-button (31) which moves between a first blocking or partially blocking position of the entry of said conduit for the choice of a first low feed speed and at least a second opening position of the entry of said conduit in order to choose a second higher medicament feed speed. Said positions are defined by stop means (35, 36) and between one and the other, one or more additional intermediate positions can be provided. Furthermore, the selection push-button can be associated with an additional valve (132) in order to vary the flow of ambient air from outside during functioning at low speed.

This invention concerns the sector for apparatus for the nebulisation of a remedy for aerosol therapy, and refers in particular to an ampoule nebuliser to be used with said apparatus and provided with a nosepiece, a mask or a nasal adapter for the inhalation of a nebulised medicament on the part of the patient

Various embodiments and configurations of ampoules for nebulisation systems of a medicinal liquid to be inhaled to cure pathologies or respiratory system illnesses are already well known.

The type of ampoule nebuliser taken into consideration usually comprises, also corresponding to the preamble of claim 1:

a body that can be made up of two or more complementary elements and that define a chamber designed to contain a medicinal liquid to b e nebulised,

a conduit extending upwards from the bottom of said chamber, designed for the input of a flow of compressed air, terminating with a top orifice and in addition a nebuliser nozzle possibly overlooked by a jet atomizer cylinder, generally called an atomizer or “pisper”,

a through conduct, open both towards the external and towards the nebuliser nozzle for the feed of an additional flow of ambient air needed to increase the delivery speed of the remedy, and

a delivery conduit that extends from said chamber towards the outside to deliver the atomised medicament towards a patient.

Compared to the longitudinal axis of the ampoule, the output conduit of the medicament can be vertical or sloping and protruding from a side of the body of the ampoule, and to it may b e attached a nosepiece, a mask or a nasal adaptor to improve the inhalation of the medicament by the patient.

The mixing and the at omizing of the liquid medicament with the air takes place by means of the nebuliser nozzle. Here, a flow of air under pressure, coming from a compressor, which arrives from the bottom to the conduit directed upwards in the chamber, contributes by the Venturi effect to the aspiration of the medicament from said chamber and to its atomization on a level with the “pisper”.

It is well known that in said ampoule nebulisers, by varying the flow of ambient air that enters from outside through the delivery conduit, it is possible to adjust the delivery speed (if not also the size of the particles) of the medicament, the delivery speed being higher when, under the same conditions of the air under pressure supplied by the compressor, a greater flow of ambient air is added on entering.

However, according to the state of the technique some ampoule nebulisers are without any means able to vary the input of ambient air and t the atomization speed of the medicament. At the most, it is however possible to vary the input flow of ambient air by placing a finger on the entrance of the feed conduct with the drawback however of an adjustment that is highly aleatory.

Some other ampoule nebulizers have been provided, on a level with the ambient air feed cannel, with a throttle type valve that, however, can be opened in different but not definable sizes, with the disadvantage that the adjustment of the atomization speed becomes fortuitous.

Other ampoule nebulisers have been provided with a valve, or better with a cap, which is applied to the entrance of the feed channel when it is required to b e blocked so as t o lessen the amount of ambient air and to maintain the atomization speed at a minimum and which is completely removable so as to fully open the feed channel and maximize the automation speed of the medicament. Such a valve is not however easy to use and even if and when, as has taken place, it is connected to the body of the ampoule, it cannot be excluded that it may come loose and get lost.

The documents U.S. Pat. No. 5,875,774, EP 0620021, EP 0627266, EP 1234591, and WO 2003/086081 are indicative of the state of the technique.

The principal object of this invention is to avoid the inconveniences and drawbacks of the known techniques and the refore to propose an ampoule nebuliser having at least two well defined supply speeds of the medicament controlled by a means easy to adjust, able to undertake at least two or more distinct positions each corresponding to a supply speed and which however remains constrained to the body of the ampoule.

Another object of the invention is provide an ampoule of the type and for the above mentioned use and equipped to allow an adjustment of the flow of medicament even at a very low working speed and to advantageously limit dispersion of medicine.

A further object of the invention is to propose an ampoule nebuliser for more efficient devices for aerosol therapy able to use the remedy medicament better for greater efficiency in the treatment of a patient.

According to a first aspect of the invention, such objects are achieved with an ampoule nebuliser according to the preamble of claim 1 and provided furthermore, on a level with the entry of the ambient air feed conduct, with a selection push-button moveable at least between a first extreme obstruction position of the entry of said conduct, for t he choice of a first mobilization speed and a second open position of said conduit for t he choice of a second delivery speed of the medicament.

Preferably and advantageously the selection push-button can also be provided for undertaking possible intermediate positions between said two extreme positions, for a partial variable opening of said conduct and a better adjustment of the delivery speed of the medicament.

In other words, the ampoule nebulizer becomes provided with a flow regulator/variator with which it will be possible to adjust the delivery speed of the medicament without substantial variations of the dimensions of the transported medicament particles or also to vary the dimensions of the medicament particles and the quantity of the fraction breathable.

According to another aspect of the invention an additional valve is associated with the selection push-button in order to increase the delivery of medicament when the atomization speed is minimum, thus ensuring the efficiency of the treatment being carried out also in this condition.

According to yet another aspect of the invention, the ampoule, provided with a flow regulator without or with an additional valve, can also be provided with an inhalation mask or mouthpiece having at least a dispersion limiter with variable width, which without influencing the inhalation and expiration of the patient makes possible however and advantageously the reduction of the dispersion of medicine in the intermediate periods between inhalation and expiration phases.

Further details will become evident from the description that follows made in reference to the attached indicative and not limiting drawings, in which:

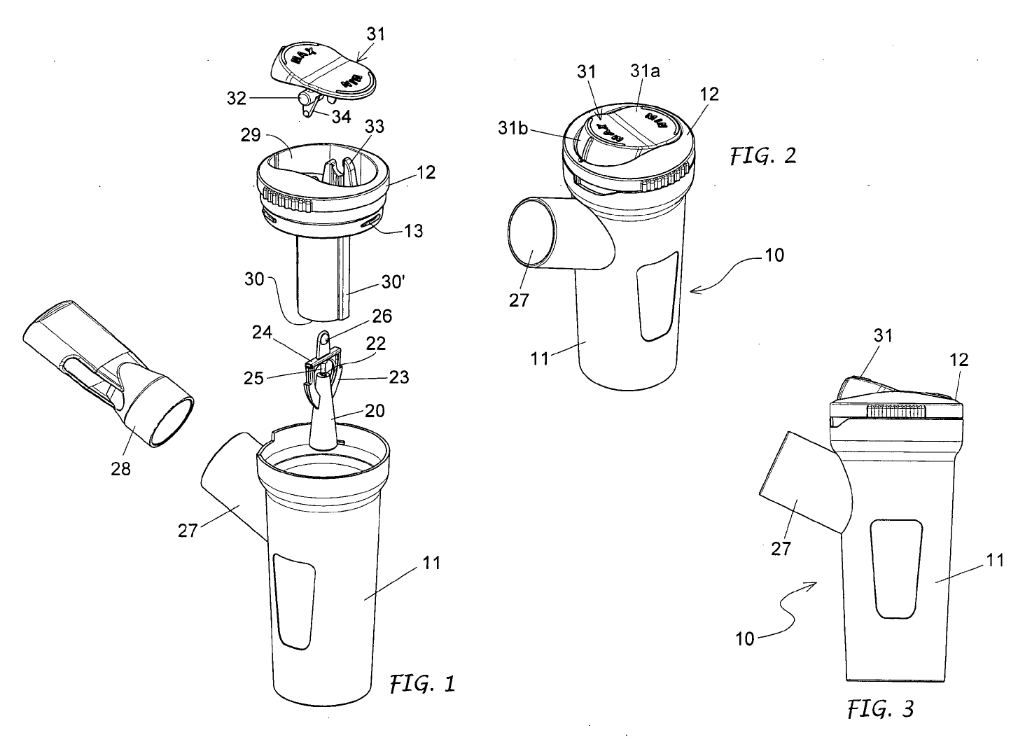

FIG. 1 shows an exploded view of the components of an ampoule complete with a speed selector;

FIG. 2 shows a view in perspective of the assembled ampoule;

FIG. 3 shows a view from a side thereof;

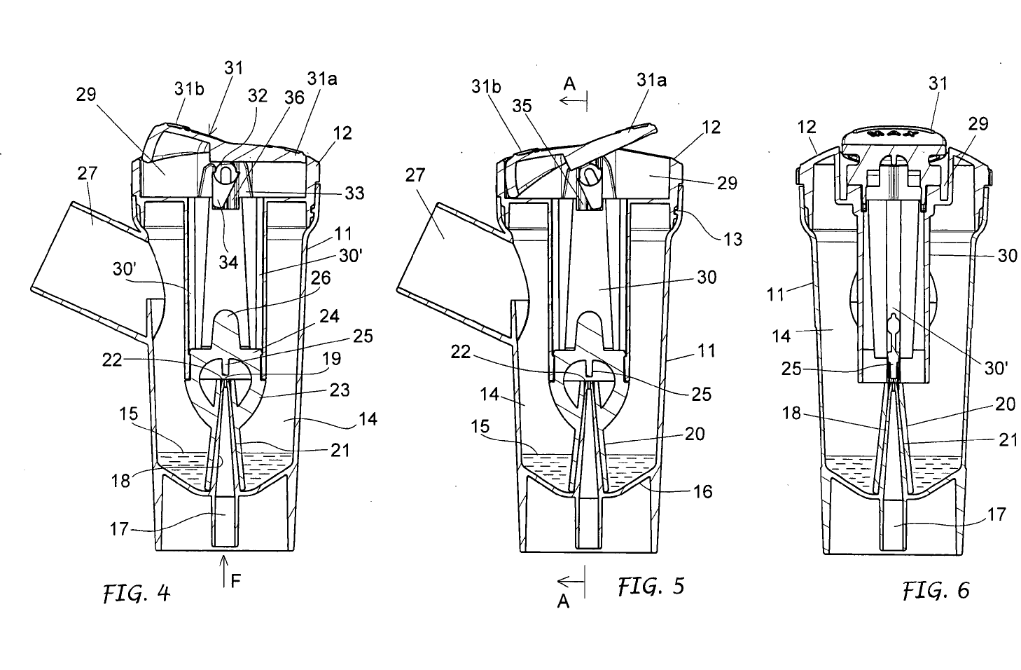

FIG. 4 shows an axial cross section of the ampoule with the selection push-button in a first position;

FIG. 5 shows a similar axial cross section, but with the selection push-button in a second position; and

FIG. 6 shows an axial cross section according to the arrows A-A in FIG. 5;

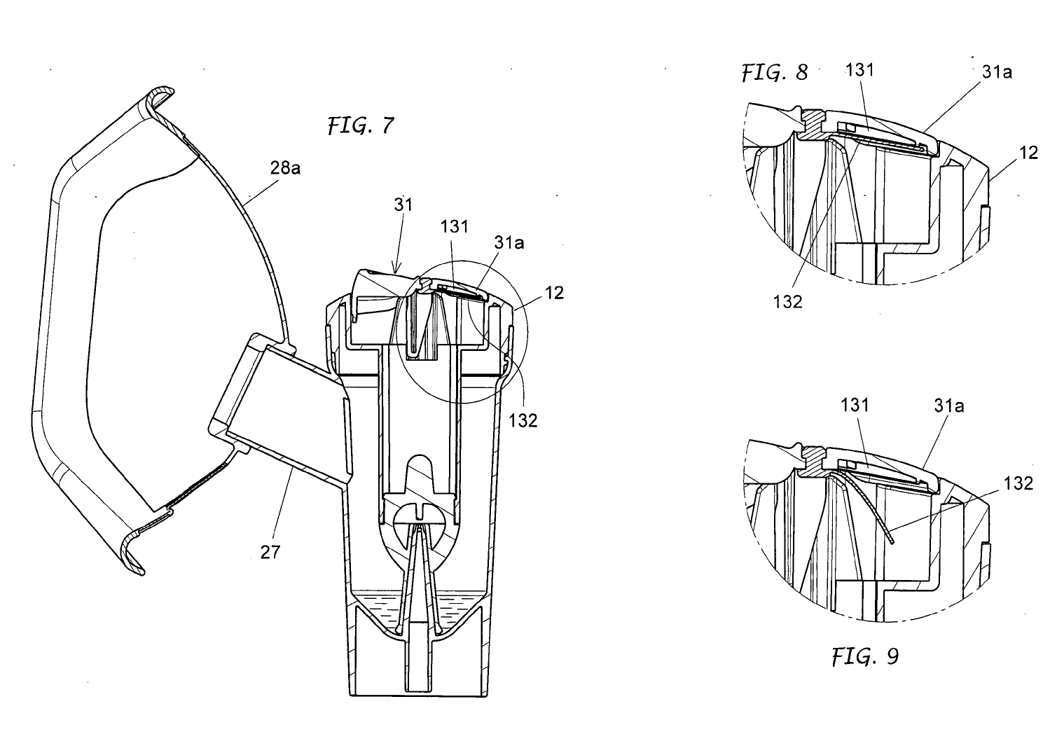

FIG. 7 shows a cross section view of an ampoule having an additional valve associated with the speed variator and also complete with a inhalation mask;

FIGS. 8 and 9 show an enlargement of the circled part in FIG. 7, with the additional valve in the closed and open positions, respectively;

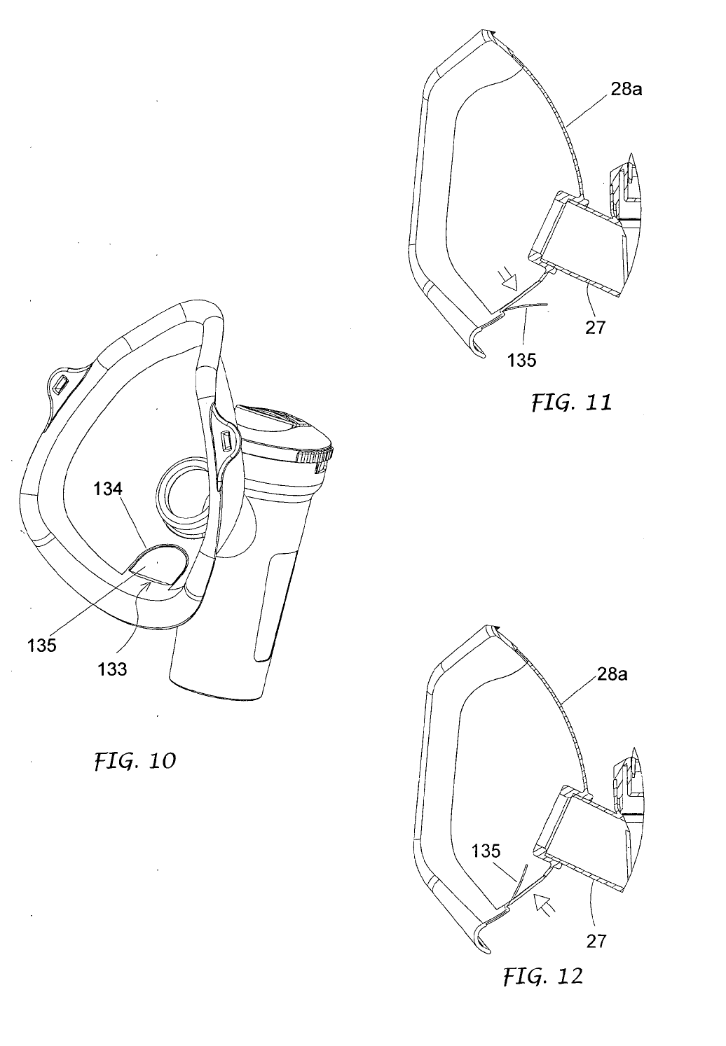

FIG. 10 shows an isometric view of the ampoule with inhalation mask as in FIG. 7; and

FIGS. 11 and 12 show the inhalation mask with dispersion limiter of the medicament in two different open positions, respectively in the inhalation and expiration phases of the patient.

The ampoule nebuliser has a body 10 made up of a lower element 11 and of a top cover 12, fixed between them in a separable form for example by means of a connection 13 by screwing or by bayonet, as shown in the drawings.

The lower element 11 delimits a chamber 14 for a liquid medicament 15 to atomize. Said chamber has a sloping bottom 16 crossed in the centre by a conduit 17 for air to enter under pressure, according to arrows F-FIG. 4, coming from a compressor, not shown. The conduit 17 has a part 18 that ascends in the chamber 14 above the level of the medicament 15 and which terminates in a conical shape (or also cylindrical) with an orifice summit 19. On the ascending part 18 of the air conduit 17 a cap 20 is fitted that delimits with said part 18 an annular cavity 21, which is open at the bottom towards the chamber 14 and dips in the medicament 15 and, at the top, flows into an injector nozzle 22 placed in line and on a level with the orifice 19 of the conduit 17. From the cap 21 two arms 23 extend upwards supporting a so-called “pisper” consisting in a beam 24 and a break-jet cylinder 25 facing towards the injector nozzle 22 and eventually an appendix 26 facing upwards.

In the example represented, the lower element 11 of the ampoule body 10 has an exit conduit 27 of the medicament from the chamber 14 towards the utilization, a mouthpiece—FIG. 1 or a inhalation mask 28a—FIG. 7— or a nose adaptor being connectable to the conduit 28, to facilitate the inhalation of the medicament on the part of a patient.

The cover 12 associated with the lower element 11 of the ampoule body 10 has an upper hollow 29 with a bottom on a level with which there is the entry of a feed conduit 30 that extends downwards in the chamber 14 as far as the proximity of the “pisper”. The conduit 30 has two radial grooves 30′ by means of which it unambiguously connects with the two arms 23 of the cap 20 defining the injector nozzle 22, so as to always guarantee the same direction of the same “pisper”.

This assembly, furthermore, facilitates the normal usage and washing operations of the ampoule carried out by the user, limiting the number of small items to be separately manipulated and furthermore easy to lose. In fact, thanks to this arrangement, from the point of view of the user, the ampoule is made up of only two parts: “body, or lower part, and cover, or upper part”.

The feed conduit 30 is designed to allow the entrance from outside of a flow of ambient air in the chamber 14. This flow of air can be varied by means of a speed regulator/variator. For this purpose, on a level with the hollow 29 of the cover, a key or selection push-button 31 is located, which is provided with pins 32 defining an oscillation axis and by means of which the push-button is assembled balancing on supporting forks 33, a Iso with the possibility of separating it for washing operations of the components of the ampoule.

The selection push-button 31 has essentially a first portion 31a and a second portion 31b on opposite parts of its oscillation axis and is moveable at least from a first position—FIG. 4— to a second position—FIG. 5— delimitated by at least a stop tang 34 integral with the push-button itself and resting, in the example shown, alternatively in two or more stop and positioning seats 35e 36.

In the first position, the selection push-button 31 obstructs by its first portion 31a the entry of the delivery conduit 30, reducing the contribution of ambient air to the chamber 14 for a first, minor, nebulization speed and delivery of medicament towards the patient through the exit conduit with mouthpiece, inhalation mask or nasal adapter. In this case, in fact, the air enters from outside and flows to the feed conduit 30, then to the chamber 14, only through the gaps around the push button, between the latter and the lateral surfaces of the hollow 29, furthermore reducing the noise of the system.

In the second position, the first portion 31a of the selection push-button 31 allows on the other hand a free access of the ambient air to the feed conduit 30 for an increase in the flow of air to the chamber 14, creating in this way the condition for a second, greater, atomization and delivery speed of the medicament towards the patient.

Furthermore, between the extreme positioning seats 35, 36 other intermediate positioning seats can be provided so as to establish as many different opening positions of the push-button 31 and by this further various delivery speeds of the medicament.

The portion 31a of the key or selection push-button 31 designed to obstruct or not allow the entry of the feed conduit 30, presents one or mo re passages 131 in line with which an additional valve 132 is associated with the push-button—FIG. 7—, for example the membrane type, that is flexible, moveable between a closed position—FIG. 8— and an open position—FIG. 9.

When the selection push button 31 is in the first position for a first, minor, nebulization speed and as long as the additional valve 132 is in the closed position as in FIG. 8, said valve has very little influence over the useful feed of medicament at that speed. However with the inhalation of the medicament on the part of the patient, the additional valve 132 tends to open as in FIG. 9, allowing in this way the air to enter from outside through the passages 131 and by this the formation of an aerosol richer in medicament even if the selection push-button remains in the first position corresponding to the lower running speed of the device.

When on the other hand the selection push-button 31 is in the second higher nebulization and medicament feed speed position the additional valve remains completely inactive.

As said above, to the exit conduit 27 of the medicament from the chamber 14 of the ampoule either a mouthpiece 28 or a inhalation mask 28a or a nasal adapter is applicable. According to the invention at least one of these parts, in particular the inhalation mask 28a, can be made of a bi-material and provided with at least a dispersion limiter means 133 with the function of limiting the dispersion, that is to say a loss of medicament towards the external of the half mask.

In the example shown, said dispersion limiter means 133—FIG. 9— is in the form of a n opening variable in width defined by at least a split 134 provided in at least one thin zone of the wall of the inhalation mask 28a so as to delimit at least a flexible portion 135, which starting from a coplanar arrangement to the surrounding wall of the inhalation mask it can bend both towards the inside and towards the outside of the mask itself. Should there be a lack o f stress, the flexible elastic portion 135 remains in that coplanar position, which corresponds to the width of the opening 133 which is null or nevertheless minimal. In the course of a treatment, during the inhalation and expiration phases of the patient, flexions are produced in the elastic flexible portion 135 towards the inside and outside of the inhalation mask, respectively, with a corresponding variation in the width of the opening as shown in FIGS. 11 and 12, respectively. In the intermediate phases between inhalation and expiration the elastic-flexible sector 135 places itself in a coplanar position to the surrounding wall of the inhalation mask, limiting in this way the dispersion of medicament.

To be noted that when the selection push-button 31 of the ampoule is provided with an additional valve 132 there must b e a correlated rigidity between that valve and dispersion limiter means 133 in the sense that work wise the valve 132 on the selection push-button has to open before the dispersion limiter means 133.

1. An ampoule nebulizer for aerosol therapy devices, comprising:
an ampoule body defining a chamber for containing a medicament liquid to be nebulized;
a conduit extending upwards from a bottom of said chamber for an input of a flow of compressed air, said conduit terminating with a top orifice and bearing a nebulizer nozzle;
a through conduit open both towards an external environment and towards the nebulizer nozzle for an additional flow of ambient air; and
a delivery conduit extending from said chamber to deliver nebulized medicament towards a mouthpiece or an inhalation mask or a nasal adapter for use on a part of a patient, wherein a selection push-button is provided on a level with an entry of said through conduit said selection push-button being movable between a first operating position obstructing at least partially the entry of said through conduit for a first minimal atomization speed and at least a second operating position opening the entry of said through conduit for the a second higher atomization speed of the liquid medicament, said first operating position and said second operating position being defined by blocking means, said ampoule body comprising a lower element and a top cover connected to each other by screwing or bayonet, said cover having an upper hollow portion and said through conduit extending from a bottom of said upper hollow portion downwards to a proximity of said nebulizer nozzle, said selection push-button being mounted and removable in said upper hollow portion above the entry of said through conduit and said selection push-button being accessible on a level with said cover.

2. An ampoule nebulizer according to claim 1, wherein the selection push-button has a portion for obstructing at least partially the entry of said through conduit provided with one or more passages for entrance of ambient air, said selection push-button being associated with an additional valve movable between a closed position and an open position of said one or more passages when the selection push-button is in said first operating position and in response to every inhalation phase of the patient, said nebulizer nozzle being overlooked by a break-jet cylinder.

3. An ampoule nebulizer according to claim 1, wherein said delivery conduit is associated with the inhalation mask provided with at least a limiter means for reducing dispersion of the nebulized medicament towards the external environment at least between inhalation and expiration phases of the patient.

4. An ampoule nebulizer according to claim 2, wherein said additional valve comprises at least an elastic-flexible membrane.

5. An ampoule nebulizer according to claim 3, wherein said limiter means comprises at least a variable width opening defined by at least a split provided in at least a thin zone of a wall of the inhalation mask so as to delimit at least a flexible portion which is normally coplanar to said thin zone of the wall of the inhalation mask for a minimum width of the opening and flexible both towards the inside and towards the outside of the inhalation mask so as to vary the width of the opening in response to the inhalation and expiration phases of the patient.

6. An ampoule nebulizer according to claim 1, wherein between said first operating position and said second operating position of the selection push button one or more intermediate opening positions are established by respective stopping means so as to create further atomization speeds for delivery of the nebulized medicament.

7-8. (canceled)

9. An ampoule nebulizer according to claim 1, wherein said selection push-button has a stop tang selectively engaging with said blocking means to define said first operating position, said second operating position and corresponding different atomization speeds of the liquid medicament.

10. An ampoule nebulizer according to claim 1, wherein, in said first operating position, the selection push-button at least obstructs partially the entry of the through conduit, reducing both a contribution of ambient air to the chamber for a first, low, output atomization speed of the liquid medicament, and noise of the ampoule device, the air entering from the external environment and flowing in the through conduit, consequently to the chamber, only through interstices between the selection push-button and a lateral surface of the upper hollow portion, and wherein in at least said second operating position the selection push-button allows free access of the ambient air to the through conduit (30), for a higher flow of air to the chamber and at least a higher atomization speed of the liquid medicament.

11. An ampoule nebulizer according to claim 1, wherein the entry of the through conduct has two radial grooves for receiving two opposite parts of a means defining the nebulizer nozzle for a precise and univocal positioning of the nebulizer nozzle in said chamber.
